(19) United States
(12) Patent Application Publication
(10) Pub. No.: US 2018/0051349 A1
(43) Pub. Date: Feb. 22, 2018

(54) Method for Obtaining a Superoxide Dismutase (SOD)-Concentrated Protein Extract

(71) Applicant: Universite de Bretagne Occidentale, Brest (FR)

(72) Inventors: Christian Magne, Brest (FR); Karim Ben Hamed, Ariana (TN)

(73) Assignee: Universite de Bretagne Occidentale, Brest (FR)

(21) Appl. No.: 15/540,723

(22) PCT Filed: Dec. 28, 2015
(86) PCT No.: PCT/EP2015/081303

§ 371 Date: Nov. 8, 2017

(30) Foreign Application Priority Data
Dec. 29, 2014 (FR) 1463385

(51) Int. Cl. A61K 36/18; C12N 9/02; C07K 1/14; A61K 38/44; A23L 3/3571; A23L 33/185; A61K 8/64; A61Q 19/08

(52) U.S. Cl. CPC C12Y 115/01001; C12N 9/0089; C07K 1/145; A61K 36/18; A61Q 19/08; A23L 3/3571; A23L 33/185; A61K 8/645; A61K 38/446

(57) ABSTRACT

The invention relates to a method for obtaining a superoxide dismutase (SOD)-concentrated protein extract having a specific activity of at least 700 UI/mg protein, said method comprising a single step of extraction from a plant selected from any species of the Plumbaginaceae family.

The present invention relates to a method for obtaining a superoxide dismutase (SOD)-concentrated protein extract, the extract obtained by this method, and the use of same.

The present invention also relates to a composition comprising the extract obtained by the method of the invention.

Superoxide dismutases (SODs) are enzymatic proteins that have a powerful antioxidant action against the reactive oxygen species (ROS) generated in living organisms (animals and plants) by any type of environmental stress or during natural aging. Catalyzing the removal of the first oxygen species formed (superoxides) following the reduction of oxygen, SODs thus constitute the first line of defense against these ROS, which are strongly implicated in various neurodegenerative diseases (Alzheimer's disease, Parkinson's disease), atherosclerosis, rheumatoid arthritis, Crohn's disease and certain cancers (Campana et al., 2004; Valdivia et al., 2009).

SODs are metalloproteins, which contain Cu/Zn, Mn or Fe atoms in their active site. These metals play an important role in the mechanism of action of SODs. For example, for Cu/Zn-SOD, Cu²⁺ is first reduced by the superoxide radical (O₂^{.-}) which is thus detoxified to oxygen. The reduced copper then gives up an electron to a new superoxide molecule that, in the presence of two protons, generates H₂O₂, which is much less toxic than O₂^{.-}.

Because of their antioxidant capacity, SODs are widely used in various fields:
In cosmetics, SODs protect the skin and hair by maintaining the integrity of the natural keratin structure (FR 7331354).
Experiments carried out at the Besancon University Hospital (France) have shown that SOD supplementation prepares human skin for sun exposure (Annual Congress for Dermatological Research, Brest, France, May 2005).
In the pharmaceutical industry, medicinal formulations based on SOD-rich extracts (e.g., orgotein) have been proposed to lower the level of the ROS strongly implicated in various neurodegenerative or inflammatory diseases.
In addition, a bioactive vegetable formula for oral administration, consisting of a melon (Cucumis melo) extract rich in SOD (Vouldoukis et al., 2004) coupled to gliadin, a protein extracted from wheat (Menvielle-Bourg, 2005), has been proposed: superoxide dismutase gliadin (GliSODin) (FR 2729296). This dietary supplement is particularly suitable for combating free radical overproduction, notably when the body's natural defenses are weakened: old age, sun exposure, tobacco use, stress, intense physical exercise, etc. It prevents certain chronic oxidative stress-related ailments or slows their progression, thus improving the patient's quality of life.
In the food industry, SOD-rich vegetable extracts (e.g., PROMUTASE™ 200, based on melon) are incorporated into feed for sport animals such as racehorses or carrier pigeons to increase their resistance to intense effort. The same extracts are incorporated into feed before and during the transport of farmed fish and shellfish (shrimp, fry, trout, salmon, etc.) in order to reduce the mortality rate during such transport.
To improve the quality of the milk produced, SOD-rich extracts (e.g., PROMUTASE™ R) are introduced into the feed of dairy-farm females (cows, goats, etc.).
SOD-rich extracts go into certain feed formulations to improve the growth performance of meat chickens and turkeys, animals often confronted with poorly controlled digestive imbalances.

SODs can be of very diverse origins: animal (for example bovine or porcine), human (for example placental or blood), microbial (for example bacteria or yeasts), etc.

They can also be derived from genetic engineering or chemical synthesis.

Several SODs are of plant origin (fungi, algae, spinach, cereals, fruits and vegetables, etc.).

Among the various plants, a melon variety (Cucumis melo) is rich in SOD (Vouldoukis et al., 2004). This melon variety is characterized by resistant seeds that have a high germination capacity, and by fruits with slowed aging (protected from the effects of free radicals).

From the above, it is clear that the role of SODs in reducing oxidative stress is very important.

Moreover, in view of its wide use in various industrial fields, there is a very great interest in finding new hyperproductive natural sources of this high specific-activity protein.

Because SOD is a fragile protein, it is necessary to have methods for obtaining it in large quantities or, still better, in smaller quantities but with a high specific activity, without increasing the use of chemical compounds capable of altering the functionality of this protein and decreasing its specific activity.

SOD can be obtained simply by protein extraction from plants comprising it in large quantities and having a high specific activity. It is preferable that such SOD extraction methods do not involve the use of a multitude of solvents, notably organic solvents, which require the implementation of additional purification steps and degrade a portion of the SOD.

Moreover, it should be noted that, in most cases, SOD is extracted from plants using extraction methods comprising an SOD-enrichment step. The currently-known SOD extraction methods require several steps to be carried out before a highly SOD-concentrated extract having a high specific activity is obtained. Such a method is described for example in the patent application FR 2 747 044, said method comprising, in addition to the extraction step, a step of purifying the filtrate obtained by extraction with a precipitation agent for increasing the specific activity of SOD in the final extract. The enzymatic activity of the SOD in the extract after the extraction step is 1482 IU/g. This extract is obtained from cereal plants, leguminous plants and oleaginous plants.

Thus, the main disadvantages of these multistep methods are their high cost and their often-long execution times. In particular, to obtain an SOD having a high specific activity, an additional purification step typically including a precipitation step is systematically performed. Thus, these multistep methods are difficult to adapt to a large scale and often require the use of several chemical reagents, including solvents, which must be removed from the extract, which is not always easily done and increases the method's cost.

There is thus a need to have new methods for obtaining high specific-activity SOD, the implementation of which is simple and fast and the cost of which is low. Besides the high specific-activity SOD, the extracts obtained must be clean, i.e., free of the chemical reagents necessarily used during the extraction process, which will make it possible to optimize the use of these extracts notably in human food and animal feed. It is also desirable that the extract obtained by these methods contains other beneficial active substances naturally present in plants, such as polyphenols, which have antioxidant properties.

The purpose of the present invention is thus to implement a method for extracting very high specific-activity SOD. This method is simple, fast, inexpensive; it produces an extract free of chemical reagents and containing other natural substances having a beneficial effect; and it thus overcomes the disadvantages of the SOD extraction methods known in the prior art.

The Inventors discovered that, surprisingly, the protein extracts obtained from species of a family of coastal plants are so rich in SOD activity that a simple aqueous extraction from the ground plant, without addition of other solvents (alcoholic solvents, for example) and without additional purification steps, is sufficient to obtain extracts where the specific activity of SOD is at least 700 IU/mg protein. This level greatly exceeds the highest level (126 IU/nng) reported in the literature to date for a melon variety (Vouldoukis et al., 2004) regarded as a reference among the natural sources of SOD.

Thus, the Inventors implemented a simple, low-cost SOD extraction method that makes it possible to obtain highly SOD-active protein extracts free of the chemical reagents typically used in conventional extraction methods and containing other natural substances having a beneficial effect, thus overcoming the disadvantages of the SOD plant-extraction methods of the prior art.

A first object of the present invention thus relates to a method for obtaining a superoxide dismutase (SOD)-concentrated protein extract having a specific activity of at least 700 IU/nng protein comprising a single step of protein extraction from a plant selected from any species of the family Plumbaginaceae.

In the context of the present invention, the expression “extraction method” or “extraction” refers to the implementation of a set of steps for obtaining a protein extract from a source, preferably a plant source, said steps comprising at least one step of collecting a liquid phase after contacting (under suitable conditions) the solvent with said source, then a purification step comprising a precipitation step, in order to obtain the protein extract sought.

Thus, in the context of the present invention, the expression “in a single extraction step” means that a single step is carried out among the steps cited above, namely collecting the liquid phase after contacting the solvent with the source, preferably the plant source. Preferably, the protein extraction method according to the present invention thus does not comprise steps of purification of the SOD protein and/or steps of enrichment in the SOD protein.

In the context of the present request, the term “specific activity” refers to SOD activity in relation to amount of protein, i.e., SOD activity in the protein extract.

The Inventors discovered that, on average, the extracts obtained directly (with no purification and/or enrichment steps) by a single step of contacting water with ground plants of the family Plumbaginaceae, a family of plants common to coastal regions, have a specific activity of at least 700 IU/mg protein. In particular, this specific activity can be at least 1000 IU/mg, more particularly at least 1200 IU/mg and even more particularly at least 1400 IU/mg.

In the context of the present invention, the abbreviation “IU” refers to SOD enzyme units in the international system. The SOD enzyme unit is defined by McCord and Fridovich (1969).

Particularly, the extracts obtained by the method of the present invention are concentrated in SOD having a specific activity ranging between 700 and 2000 IU/mg protein, more particularly between 1000 and 1800 IU/mg protein, even more particularly between 1200 and 1600 IU/mg protein and more particularly still between 1300 and 1500 IU/mg protein.

The Inventors carried out comparative tests (see comparative examples) that show that other coastal species belonging to the families Apiaceae, Brassicaceae, Aizoaceae and Chenopodiaceae have an SOD specific activity ranging between 20 and 400 IU/mg, that is to say much lower than that of species of the family Plumbaginaceae.

In the context of the present request, the term “catalytic activity” refers to SOD activity in relation to the source plant biomass.

This catalytic activity is, in species of the family Plumbaginaceae, in a range between 3000 and 14000 IU/g DM (activity units/g dry matter), particularly between 5000 and 13000 IU/g DM and even more particularly between 7000 and 12000 IU/g DM.

The Inventors also carried out comparative tests (see comparative examples) that show that the species of the families Apiaceae, Aizoaceae, Brassicaceae and Chenopodiaceae have an SOD catalytic activity ranging between 120 and 1250 IU/g DM, that is to say much lower than that of species of the family Plumbaginaceae.

The family Plumbaginaceae is a cosmopolitan family of dicotyledonous plants. The species of this family are present everywhere, particularly in cold, saline environments where their ability to adapt gives them a selective advantage.

In France, the family Plumbaginaceae is represented by the three genera Plumbago, Limonium and Armeria. Of these, the genera Limonium and Armeria are the most commonly encountered of this family. They are found everywhere on the coast, from the English Channel to the Mediterranean, by way of the Atlantic coast.

Thus, according to a preferred embodiment of the method of the invention, the plant from which the SOD is extracted is selected from the group consisting of the genera Limonium and Armeria.

Advantageously, the method of the invention is carried out using species of the genera Limonium and Armeria encountered everywhere on the coast of France, preferably on the Atlantic coast of France.

The genus Armeria contains a single species, which is named Armeria maritima.

The genus Limonium comprises hardy herbaceous plants with purple flowers which grow on the sandy or poor soils of the Atlantic coast and in salt-water marshes.

The species of the genus Armeria are hardy perennials of the coastal dunes and rocks which flower from July to September.

According to an even more preferred embodiment of the method of the present invention, the extraction of SOD is carried out from plants selected from the group consisting of the species Limonium latifolium, Limonium normannicum, Limonium vulgare, Limonium tunetanum, Limonium densiflorum, Limonium pruinosum, Limonium delicatulum, Limonium spathulatum, Limonium boitardii, Limonium wrightii and Armeria maritima.

All these species are very rich in SOD having a very high specific activity that is higher than that of the SOD extracted from the organisms used to date as a source of SOD.

Moreover, these species have the advantage of being easy to cultivate in open fields, which is unusual for species whose natural environment is characterized by high salinity. In addition, species of the family Plumbaginaceae are easy to cultivate under controlled conditions (in a greenhouse), which makes their use on an industrial scale possible, indeed even easy, in any season.

Advantageously, the SOD extraction method is carried out using the species Limonium latifolium, which is easy to cultivate or access in the region where the present invention was implemented.

Indeed, Limonium latifolium, or sea lavender (also called statice), is a hardy herbaceous plant that grows on the sandy or poor soils of the Atlantic coast and in salt-water marshes. Sea lavender has antioxidant properties due to its phenolic compounds and contains coloring agents (flowers and roots).

The species Limonium latifolium has much higher SOD specific activity than the plant species used to date in the agri-food industry as sources of SOD. This means that it is not necessary to have a large amount of the plant to obtain a highly enzymatically-active SOD, whence a lower cost of producing an SOD-enriched end product.

Indeed, the specific activity of SOD in the extracts obtained from this species exceeds 1200 units/mg protein, that is to say a level much higher than the specific activity of SOD in the extract obtained from melon, the species hitherto considered the richest in SOD (Vouldoukis et al., 2004).

Moreover, the species L. latifolium is a species that is easy to cultivate, with a faster development cycle than melon, with no known predator, whence the possibility of producing this plant under conditions consistent with regulations concerning organic crops.

Lastly, given that Limonium latifolium is a halophytic, and thus salt-resistant, species and that salt stress is known to stimulate the synthesis of antioxidant compounds including enzymes (Jithesh et al., 2006; Ben Hamed et al., 2007, Li et al., 2008, Hameed et al., 2014), it can be envisaged to further amplify the SOD activity in the organs of this plant by cultivation under controlled conditions in the presence of salt. Thus, it will be possible to produce that under low-cost conditions while preventing any development of harmful organisms (weeds, pathogens, etc.) sensitive to salt.

The Inventors thus carried out further tests that show that SOD activity can be increased by 10% to 20% in plants of the species L. latifolium cultivated in the presence of salt at a concentration of 20 g/L NaCl (or ⅔ of the salt concentration in seawater).

Among the species of the family Plumbaginaceae studied, the Inventors have shown that the species Limonium latifolium has particularly desirable properties and advantages as described above. The Inventors also demonstrated that other species of the genus Limonium have these same properties and advantages and can thus be easily cultivated under controlled conditions in the presence of salt in order to increase the specific activity of SOD.

According to a preferred embodiment of the method of the present invention, the extraction is carried out from the aerial parts of the plant, preferably from the leaves, which are the fleshiest organs and the richest in SOD.

According to a preferred embodiment, the method of the invention comprises a single extraction step consisting in collecting an aqueous phase containing the plant extract after contacting the ground plant of the family Plumbaginaceae with water. Particularly, the Inventors have shown that plants of the family Plumbaginaceae are so rich in SOD that simple aqueous extraction without addition of other solvents or chemicals makes it possible to obtain an extract whose SOD specific activity is at least 700 IU/mg protein.

According to a particularly preferred embodiment, in the method of the present invention, the extraction is carried out in the presence of water or a phosphate buffer-type aqueous buffer, thus in the absence of alcohol-type toxic organic solvents (e.g.: ethanol, methanol, propanol, isopropanol or butanol), aromatic solvents (e.g.: pyridine, toluene, chlorobenzene or xylene), carbonyl-containing solvents (e.g.: acetone, dimethyl formamide or methyl pyrrolidone), esters (e.g.: ethyl acetate), sulfoxides (e.g.: dimethyl sulfoxide), nitrites (e.g.: acetonitrile), halogenated solvents (e.g.: dichloromethane, dichloroethane, chloroform, carbon tetrachloride, chlorobenzene), ethyl oxides (e.g.: diethylene glycol, tetrahydrofuran, ethyl ether).

In certain cases, glycerin, which acts as a stabilizer, is added during the extraction.

According to an embodiment, before the extraction step, said plant can be washed, cut, ground, micronized and sieved.

According to another embodiment of the invention, the extraction can be carried out from plant matter that has been frozen or dried beforehand so as to enable it to be preserved and/or stored. Preferably, the plant is dried by lyophilization in order to avoid degradation of the enzyme.

A second object of the present invention relates to a plant protein extract concentrated in SOD having a specific activity of at least 700 IU/mg protein, the extract being obtained by the method of the invention as defined above.

The extract obtained according to the method of the invention has all or some of the characteristics of said method as described below.

In particular, the extract obtained by the method of the invention is an extract not containing the chemical reagents typically used during conventional extraction methods and whose removal is frequently linked to a decrease in the quality of the extract since it leads to a lower specific activity of SOD and to the destruction of certain substances naturally present in the plant and having a beneficial effect.

In order to preserve the enzymatic activity of SOD in the extract of the invention, according to a particular embodiment, the extract of the invention can be stored in the cold, i.e., at a temperature between 4° C. and −30° C., preferably between −2° C. and −30° C., more particularly between −18° C. and −27° C.

According to another embodiment, the extract can be preserved by drying, preferably drying by lyophilization.

Thus, the extract of the present invention may comprise other substances naturally present in species of the family Plumbaginaceae and having a beneficial effect, such as polyphenols, which are known for their antioxidant properties.

According to a particular embodiment, the extract of the invention contains predominantly the Cu/Zn SOD isoform.

The extract of the present invention has the advantage of being very rich in SOD activity and other antioxidant substances, such as polyphenols, and, due to the method by which it is obtained, of containing none or very little of the chemical reagents typically used during extraction.

The extract of the invention is thus perfectly suited for use in human and/or animal medicine and/or cosmetics.

A third object of the present invention thus relates to a cosmetic or pharmaceutical composition comprising the extract obtained according to the method of the present invention and a cosmetically or pharmaceutically acceptable carrier, respectively.

By “pharmaceutically acceptable carrier” is meant, in the context of the present invention, any substance suitable for use in a pharmaceutical product. Examples of pharmaceutically acceptable carriers include lactose, optionally modified starch, cellulose, hydroxypropylmethyl cellulose, mannitol, sorbitol, xylitol, dextrose, calcium sulfate, calcium phosphate, calcium lactate, dextrates, inositol, calcium carbonate, glycine, bentonite and mixtures thereof.

Preferably, the pharmaceutically acceptable carriers are selected from the group consisting of prolamins, i.e., storage proteins accumulated by plants of the family Poaceae, such as cereals.

The pharmaceutical composition and the medicinal products according to the invention can be in various forms, in particular in a form selected from the group consisting of injectable solutions, tablets, gelatin capsules, sugar-coated pills, syrups, suspensions, solutions, powders, granules, emulsions, microspheres. In addition, extended-release dosage forms such as gelatin capsules, or tablets optionally coated for extended release, will be preferred.

The pharmaceutical composition and the medicinal products according to the invention can be administered by various routes. Examples of administration routes that can be used for the pharmaceutical composition and the medicinal products according to the invention include the oral, rectal, cutaneous, nasal, sublingual, parenteral notably intradermal, subcutaneous, intramuscular, intravenous, intra-arterial, intra-articular, intrapleural and intraperitoneal routes.

The pharmaceutical composition and the medicinal products according to the invention can be administered one or more times or in continuous release. Preferably, the pharmaceutical composition or the medicinal products according to the invention are administered in continuous release, such as in the form of a perfusion or by means of an extended- or delayed-release dosage form, such as a capsule or an optionally-coated tablet.

In the context of the present invention, by “cosmetically acceptable carrier” is meant a carrier that is suitable for use in contact with human and animal cells, in particular epidermal cells, without being toxic, irritating or provoking an allergic response, and that is proportioned in a reasonable advantage/risk ratio.

The cosmetic composition according to the invention may comprise one or more formulating agents or additives commonly and traditionally used in cosmetic and dermatological compositions such as, by way of non-limiting example, emollients, colorants, film-forming agents, surfactants, fragrances, preservatives, emulsifiers, oils, glycols, vitamins such as vitamin E, UV filters, etc. Using their knowledge of cosmetics and/or dermatology, persons skilled in the art will know what formulating agents to add to the compositions of the invention and in what amounts according to the desired properties.

The cosmetic composition according to the invention may be in any form known to persons skilled in the art in the field of cosmetics and dermatology with no pharmaceutical restriction other than application on the face and body. Advantageously, the compositions according to the invention are in the form of a gel, a cream, a lotion, an oil, a milk, a spray, etc.

Whatever the objective, a once- or twice-daily application for a period of several days to several months may be proposed.

Since the extract of the invention can be obtained by aqueous extraction without the use of other chemical reagents (alcohols, organic solvents, buffers), it is particularly suitable for use in human food and animal feed.

Thus, the present invention further relates to a dietary or nutraceutical composition comprising a protein extract obtained according to the method of the invention and a food additive or a nutraceutically acceptable carrier, respectively.

The food additives may be selected from all the food additives well-known to persons skilled in the art, for example from the group consisting of preservatives, food colorants, antioxidants, lactates, citrates, orthophosphates, malates, adipates, alginates, gums, diphosphates and triphosphates, etc.

By “nutraceutical composition” is meant, in the context of the present invention, a composition relating to a product produced from food and marketed in tablet, powder or potion form having a beneficial physiological effect against chronic diseases.

By “nutraceutically acceptable carrier” is meant, in the context of the present invention, any substance that is suitable for use in a nutraceutical product. Examples of nutraceutically acceptable carriers include dicalcium phosphate, calcium alginate, magnesium carbonate, magnesium stearate, silicon dioxide, calcium chloride, etc.

The nutraceutical composition according to the invention can be administered notably orally, for example, in the form of tablets, which may be scored or film-coated, granules, capsules, gelatin capsules, or in the form of loose powders packaged preferably in unit-dose sachets, or compressed powder.

When the extract obtained by the method of the invention is intended for use in a dietary and/or nutraceutical composition, it is preferable to carry out an aqueous extraction by implementing the method of the invention and not an extraction using reagents likely to cause discomfort and/or a digestive disorder, such as protease inhibitors and/or phenol-reducing or -binding agents, for example.

According to an embodiment, said extract comprises about 50,000 to 200,000 IU/g dry extract, preferably about 100,000 to 150,000 IU/g, or, by weight, around 1% to 4%, preferably 2% to 3% superoxide dismutase in the dry extract.

By “dry extract” is meant, in the context of the present invention, the dry residue remaining after an extract is dried, for example by vacuum evaporation at 40° C. or by lyophilization.

Moreover, the composition according to the invention may comprise from 0.1% to 20%, more particularly from 0.1% to 10% or from 0.5% to 5%, in particular from 1% to 3% superoxide dismutase by weight relative to the total weight of the composition.

According to an embodiment, the pharmaceutical composition according to the invention comprises from 5% to 20%, particularly from 5% to 15% and more particularly from 5% to 10% superoxide dismutase by weight relative to the total weight of the composition.

According to a particular embodiment, the dietary composition and/or the nutraceutical composition according to the invention may comprise from 0.1% to 5%, particularly from 0.5% to 3% and more particularly from 1% to 2% superoxide dismutase by weight relative to the total weight of the composition.

A fourth aspect of the present invention relates to the cosmetic use of a protein extract as defined according to the invention as an antioxidant cosmetic agent, in particular for combating aging of the skin and/or skin appendages or for protecting against UV rays, etc.

A fifth aspect of the invention relates to the use of an SOD protein extract as defined above for preparing cheeses and/or for preserving dairy products.

In cheese production, SOD can combine with catalase and in this way effectively inhibit anarchic oxidation of the lipids in dairy products, allowing milk to be stored longer.

A sixth aspect of the present invention relates to the SOD protein extract as defined above as a drug.

In particular, the invention relates to an SOD protein extract according to the invention for use in the prevention and/or in the treatment of diseases selected from the group comprising:
psychiatric illnesses, preferably schizophrenia;
neurodegenerative diseases, preferably Alzheimer's disease or Parkinson's disease;
cardiovascular diseases, preferably myocardial infarction or atherosclerosis;
inflammatory diseases, preferably Crohn's disease or rheumatoid arthritis;
chronic diseases, preferably diabetes;
cancer diseases, preferably selected from the group comprising skin cancer, breast cancer, esophageal cancer, stomach cancer, liver cancer, colon cancer and lung cancer.

The present invention is further illustrated by the examples described below.

### EXAMPLES

### 1. Materials and Methods

1.1 Plant Material

The species tested are typically found among the sand dunes, salt-water rocks and marshes along the Brittany coast.

The species tested are Limonium normannicum, L. vulgare, L. latifolium, L. tunetanum, L. densiflorum, L. pruinosum, L. delicatulum, L. spathulatum and Armeria maritima.

1.2 Cultivation

Samples of the aerial parts of all these species were taken at the end of spring in their natural habitat.

Moreover, plants of the species L. latifolium were cultivated under the following controlled conditions: Photoperiod: 8 h/16 h (night/day); Temperature: 14/23° C. (night/day); Relative humidity: 50-70%; Salinity: 0, 5, 10, 20 g/L NaCl.

The SOD protein extraction is carried out immediately after the leaves are taken from the mother plants or after they have been preserved at −80° C. To that end, the leaves are ground in water or phosphate buffer (100 mM, pH 7.8) for 15 min at 4° C. After filtration or centrifugation at 14,000 g for 30 min at 4° C., the supernatant is collected (liquid phase). The soluble protein content and the enzymatic activities are determined from the supernatant.

1.3 Assay of Soluble Proteins

The soluble protein content is determined in the supernatant of each enzyme extract according to the Bradford (1976) method.

1.4 Measurement of SOD Activities

Superoxide dismutases catalyze the reaction:

2 O₂^{-.}(superoxide anion)+2 H+→O₂+H₂O₂

Since the substrate for SODs is highly unstable and has a very short lifespan (10⁻⁹ s), the assay methods are very indirect. The method proposed by Beauchamp and Fridovich (1969), modified by Scebba et al. (1999), evaluates SOD by its capacity to inhibit a flux of superoxide anion generated by illuminated riboflavin. The superoxide radicals produced by this system reduce nitro blue tetrazolium (NBT) to blue formazan.

Two series of tubes were prepared. The first are controls, kept in the dark and containing a mixture in 50 mM pH 7.8 phosphate buffer consisting of EDTA (0.1 mM), methionine (13 mM), NBT (75 μM), riboflavin (2 μM) and the enzyme extract. The second series of tubes is for determining SOD activity. These tubes contain the same reaction mixture but they are kept for 15 min under 15 W lighting. The absorbance measurement is carried out at 560 nm. One SOD enzyme unit corresponds to the amount of plant extract capable of inducing 50% inhibition of the nitro blue tetrazolium reduction reaction.

The catalytic activity of the enzyme is thus related to the tissue mass having been the object of the extraction whereas the specific activity, for its part, is related to the amount of proteins in the extract.

### 2. Results

The results obtained by measurements of SOD catalytic activity and specific activity in extracts obtained from plants of the family Plumbaginaceae by the method of the invention are shown in table 1 below:

TABLE 1
Results of measurements of SOD catalytic activity
and specific activity in species of Plumbaginaceae.

| | Catalytic activity | Specific activity |
|---|---|---|
| Species | (units/g DM) | (units/mg protein). |
| Family Plumbaginaceae | | |
| Limonium normannicum | 12709.40 | 1588.68 |
| L. vulgare | 10945.94 | 1368.24 |
| L. latifolium | 7812.50 | 1228.38 |
| L. tunetanum | 8051.61 | 1006.45 |
| L. densiflorum | 8836.72 | 1104.59 |
| L. pruinosum | 8027.38 | 1003.42 |
| L. delicatulum | 3703.70 | 752.78 |
| L. spathulatum | 4743.72 | 705.91 |
| Armeria maritima | 9280.74 | 1784.76 |

These results clearly show that the method for extracting SOD protein from species of Plumbaginaceae, according to the present invention, makes it possible to obtain, simply and rapidly, SOD-concentrated protein extracts having a specific activity of at least 700 IU/mg protein and a catalytic activity of at least 3000 IU/g dry matter.

### COMPARATIVE EXAMPLES

Under the same protein extraction conditions as those described above (aqueous extraction or extraction in phosphate buffer), comparative examples were prepared from halophytic plants of the families Apiaceae, Aizoaceae, Brassicaceae and Chenopodiaceae.

The results obtained are presented in table 2 below.

TABLE 2
Results of comparative tests of the catalytic activity and the specific
activity of of the SODs in species of various families halophytic plants.

| | Catalytic activity | Specific activity |
|---|---|---|
| Species | (units/g DM) | (units/mg protein) |
| Family Plumbaginaceae | | |
| Limonium normannicum | 12709.40 | 1588.68 |
| L. vulgare | 10945.94 | 1368.24 |
| L. latifolium | 7812.50 | 1228.38 |
| L. tunetanum | 8051.61 | 1006.45 |
| L. densiflorum | 8836.72 | 1104.59 |
| L. pruinosum | 8027.38 | 1003.42 |
| L. delicatulum | 3703.70 | 752.78 |
| L. spathulatum | 4743.72 | 705.91 |
| Armeria maritima | 9280.74 | 1784.76 |
| Family Apiaceae | | |
| Crithmum maritimum | 1216.55 | 405.52 |
| Eryngium maritimum | 687.62 | 172.56 |
| Family Brassicaceae | | |
| Cakile maritima | 879.31 | 314.04 |
| Crambe maritima | 1250 | 155 |
| Family Aizoaceae | | |
| Mesembryanthemum crystallinum | 160 | 20 |
| Family Chenopodiaceae | | |
| Suaeda fruticosa | 144 | 18 |
| Arthrocnemum indicum | 176 | 22 |
| Halocnemum strobilaceum | 160 | 20 |

These results clearly show that the SOD catalytic activity and specific activity of species of the family Plumbaginaceae are much higher than those of the species studied of other families.

### Bibliography

Alscher R G, Erturk N, Heath S H (2002) Role of superoxide dismutases (SODs) in controlling oxidative stress in plants. Journal of Experimental Botany 372: 1331-1341;

Bannister J V, Bannister W H, Rotilio G (1987) Aspects of the structure, function and applications of superoxide dismutase. Critical Review of Biochemistry 22: 111-180;

Beauchamp C, Fridovich I. (1971) Superoxide dismutase: improved assays and an assay applicable to acrylamide gels. Analytical Biochemistry 44(1): 276-287;

Ben Hamed K, Castagna A, Elkahoui S, Ranieri A, Abdelly C (2007) Sea fennel (Crithmum maritimum L.) under salinity conditions: a comparison of leaf and root antioxidant responses. Plant Growth Regulation 53: 185-194;

Campana F (2004) Topical superoxide dismutase reduces post-irradiation breast cancer fibrosis. Journal of Cellular Et Molecular Medicine 8 (1): 109-116;

Fox P F, Kelly A L (2006) Indigenous enzymes in milk: overview and historical aspects—Part 2. International Dairy Journal 16: 517-532;

Hameed A, Rasheed A, Gul B, Ajmal Khan M (2014) Salinity inhibits seed germination of perennial halophytes Limonium stocksii and Suaeda fruticosa by reducing water uptake and ascorbate dependent antioxidant system. Environmental and Experimental Botany 107: 32-38;

Jithesh M N, Prashanth S R, Sivaprakash K R, Parida A K (2006) Antioxidative response mechanisms in halophytes: their role in stress defence. Journal of Genetics 85 (3): 237-254;

Linden G, Desnouveaux R, Driou A (1998) Les enzymes non coagulantes dans la filière lait : propriétés, utilisations industrielles et développennents futurs. Eds Lavoisier, p. 262;

McCord J M (1999) Analysis of superoxide dismutase activity. In: Bus J S, Costa L G, Hodgson E, Lawrence D A, Reed D J (Eds.), Current Protocols in Toxicology. John Wiley and Sons, UK, Suppl. 8, pp. 731-739;

McCord J M, Fridovitch I (1969) Superoxide Dismutase. An enzymic function for erythrocuprein (hemocuprein). Journal of Biological Chemistry 244: 6049-6055;

Menvielle-Bourg F J (2005) La superoxyde dismutase, puissant antioxydant naturel, désornnais disponible par voie orale. Phytothérapie 3: 118-121;

Scebba F, Sebustiani L, Vitagliano C (1999) Protective enzymes against activated oxygen species in wheat (Triticum aestivum L.) seedlings: Responses to cold acclimation. Journal of Plant Physiology 155: 762-768.

Valdivia S, Pérez-Álvarez J D, Aroca-Aguilar I, Ikuta I, Jordán J (2009) Superoxide dismutases: a physiopharmacological update. Journal of Physiology Et Biochemistry 65 (2): 195-208;

Vouldoukis I, Lacan D, Kamate C, Coste P, Calenda A, Mazier D, Conti M, Dugas B (2004) Antioxidant and anti-inflammatory properties of a Cucumis melo LC. extract rich in superoxide dismutase activity. Journal of Ethnopharmacology 94: 67-75. EP 2 747 044

1. A method for obtaining a superoxide dismutase (SOD)-concentrated protein extract having a specific activity of at least 700 IU/mg protein, characterized in that it comprises a single step of protein extraction from a plant selected from any one of species of the family Plumbaginaceae.

2. The method according to claim 1, wherein the plant is selected from the group consisting of the genus Limonium and Armeria.

3. The method according to claim 1 or 2, wherein the plant is selected from the group consisting of the species Limonium latifolium, Limonium normannicum, Limonium vulgare, Limonium tunetanum, Limonium densiflorum, Limonium pruinosum, Limonium delicatulum, Limonium spathulatum, Limonium boitardii and Armeria maritima.

4. The method according to any one of claims 1 to 3, wherein the extraction is carried out from the aerial parts of the plant, preferably from the leaves.

5. The method according to any one of claims 1 to 4, wherein the single extraction step comprises collecting a liquid phase after contacting a solvent with said plant, said solvent preferably being an aqueous solvent.

6. The method according to any one of claims 1 to 5, wherein the extraction is carried out in the absence of organic solvents selected from the group comprising alcohols, aromatic solvents, carbonyl-containing solvents, esters, nitriles, halogenated solvents and ether-oxide solvents.

7. An SOD-concentrated plant protein extract having a specific activity of at least 700 IU/mg protein, characterized in that it is obtained by the method according to any one of claims 1 to 6.

8. The protein extract according to claim 7 comprising about 50,000 to 200,000 IU/g, preferably about 100,000 to 150,000 IU/g dry extract, or, by weight, around 1% to 4%, preferably 2% to 3% superoxide dismutase in the dry extract.

9. A cosmetic or pharmaceutical composition characterized in that it comprises an extract according to claim 7 or 8 and a cosmetically or pharmaceutically acceptable carrier, respectively.

10. A dietary or nutraceutical composition, characterized in that it comprises a protein extract according to claim 7 or 8 and a food additive or a nutraceutically acceptable carrier, respectively.

11. The composition according to claim 9 or 10 comprising from 0.1% to 20%, more particularly from 0.1% to 10%, even more particularly from 0.5% to 5%, in particular from 1% to 3% superoxide dismutase by weight relative to the total weight of the composition.

12. A cosmetic use of the protein extract according to claim 7 or 8 or of a composition according to claim 9 or 11 as an antioxidant cosmetic agent, in particular for combating aging of the skin and/or skin appendages and/or for UV protection.

13. A use of the protein extract according to claim 7 or 8 or of a composition according to claim 10 or 11 for preparing cheeses and/or preserving dairy products.

14. The protein extract according to claim 7 or 8 or the composition according to claim 9 or 11 as a drug.

15. The protein extract or the composition according to claim 14 for use in the prevention and/or in the treatment of diseases selected from the group comprising:
psychiatric illnesses, preferably schizophrenia;
neurodegenerative diseases, preferably Alzheimer's disease or Parkinson's disease;
cardiovascular diseases, preferably myocardial infarction or atherosclerosis;
inflammatory diseases, preferably Crohn's disease or rheumatoid arthritis;
chronic diseases, preferably diabetes, and
cancer diseases, preferably selected from the group comprising skin cancer, breast cancer, esophageal cancer, stomach cancer, liver cancer, colon cancer and lung cancer.
